(19) United States
(12) Patent Application Publication
(10) Pub. No.: US 2022/0257282 A1
(43) Pub. Date: Aug. 18, 2022

(54) Skin Lifting Thread Inserting Device

(71) Applicant: Doctor Biomed Co., Ltd., Gangwon-do (KR)

(72) Inventor: Ki Won Kim, Gangwon-do (KR)

(21) Appl. No.: 17/614,858

(22) PCT Filed: Jun. 24, 2020
(86) PCT No.: PCT/KR2020/008214

§ 371 Date: Apr. 18, 2022

(30) Foreign Application Priority Data
Jun. 25, 2019 (KR) 10-2019-0075537

(51) Int. Cl. A61B 17/34; A61B 17/04; A61F 2/10

(52) U.S. Cl. CPC A61B 17/3468; A61B 2017/00792; A61F 2/10; A61B 17/0483

(57) ABSTRACT

The present disclosure relates to a skin lifting thread inserting device and, more specifically, to a thread inserting device capable of enhancing a lifting effect by using a single cog thread connected to a plurality of needles. A thread inserting device for a skin lifting treatment according to one embodiment of the present disclosure comprises: first and second needles (10a, 10b); and first and second handles (20a, 20b), wherein the thread (100) extends from the opening (11a) formed at the distal end of the first needle (10a) to the opening (11b) formed at the distal end of the second needle (10b).

### CROSS-REFERENCE TO RELATED APPLICATION

This application is a Section 371 National Stage Application of International Application No. PCT/KR2020/008214, filed Jun. 24, 2020, the contents of which is hereby incorporated by reference in its entirety.

### TECHNICAL FIELD

The present invention relates to a thread inserting device for skin lifting, and more specifically, to a thread inserting device for skin lifting that is capable of enhancing a lifting effect by using a single cog thread connected to a plurality of needles.

Further, the present invention relates to a thread inserting device for skin lifting, and more specifically, to a thread inserting device for skin lifting that is capable of having a guide thread for preventing a thread wound on a needle from being easily unwound from the needle.

### BACKGROUND ART

The context described herein is not included in conventional technologies with respect to the claims of this application, unless otherwise expressly stated, and even though context is described herein, the context is not necessarily considered as the conventional technologies.

With high interest in skin care, many people become interested in skin lifting surgery capable of reducing wrinkles on the skin and keeping elasticity of the skin. In various surgery, there is a thread lifting treatment in which a thread giving no harm to a human body is inserted under the skin to thus tighten and lift up the nasolabial folds, the chin, and so on, without any operation procedure.

In specific, a thread lift is surgery that is performed by inserting a thread giving no harm to a human body into the dermis layer of the skin to improve the elasticity of sagging skin. The thread used for the thread lift is divided into a melting thread and an unmelting thread and has various characteristics of duration time and lifting effect according to thread shapes and lifting types.

A thread lift procedure is carried out by putting a thread through a thin and long needle having a given diameter hole adapted to insert the thread thereinto via the hole in a longitudinal direction of the needle, inserting the needle into a human body, by removing the needle from a desired region in a state of reaching the desired region to allow the thread to remain in the human body, and by pulling the thread, so that the skin is tightened to improve the elasticity of the skin and remove the wrinkles on the skin.

Further, a lifting thread used for the lifting surgery is classified into a straight thread, a spiral thread called whirlwind lifting thread, and a cog thread having a plurality of thorns (protrusions) formed along the middle portion thereof according to surgical regions and methods. The straight thread is adequate for the surgery for small wrinkles or thin skin, but because its area coming into contact with the subcutaneous fat is relatively small, a large number of threads are needed so as to obtain a desired lifting effect. Further, the spiral thread is a spirally twisted thread or two doubly twisted threads, thereby maximizing the area coming into contact with the tissue of skin. Lastly, the cog thread has the thorn-like protrusions formed thereon to pull the tissue of skin, and even though a relatively small number of threads are inserted, accordingly, excellent lifting effect can be created.

One of conventional thread inserting devices for inserting a lifting thread into the tissue of skin is disclosed in Korean Patent Application Publication No. 10-2017-0095162 in which a single thread is continuously inserted into a surgical region with different insertion directions.

According to the above-mentioned conventional thread inserting device, the single thread is continuously inserted into the surgical region with different insertion directions to thus allow threads to be organically inserted into the surgical region, thereby tightening muscles and reducing skin sagging or wrinkles. Through one single thread inserting needle, however, insertion and removal processes into and from the surgical region have to be repeatedly carried out several times.

Besides, after the thread has been initially inserted into the surgical region by using one thread inserting needle, it is inserted in a different insertion direction from the initial insertion direction and then removed, and in this case, the thread inserted in the second direction is unnecessarily overlaid on top of the thread inserted in the first direction, so that even in a process of pulling the thread, the threads are overlaid to undesirably cause the lifting surgery to become inefficient.

Further, another conventional thread inserting device for inserting a lifting thread into the tissue of skin is disclosed in Korean Patent No. 10-1811208 in which a thread is tightly wound on a needle, while being tied to the rear end portion of the needle. If the needle is inserted into the tissue of skin, together with the thread wound thereon, the thread insertedly remains to the shape wound on the needle.

In this case, the surgery has to be performed, without any mistake, after an insertion position or direction has to be confirmed, but in surgery using the thread inserting device requiring very accurate handling technique, it is not easy to perform the surgery with no mistake. If the insertion is erroneous, accordingly, the needle has to be removed from the inserted position. After the thread inserting device has been inserted into the surgical region, however, if the thread inserting device is removed so as to change the insertion position or direction, unfortunately, the wound thread may be unwound from the needle.

Further, in the case where the thread is wound on the needle, the rear end portion of the thread has to be tied to the needle to prevent the thread from being unwound from the needle, which causes many inconveniences in tying substantially thin thread to the needle with a tiny diameter.

### DISCLOSURE

### Technical Problem

Accordingly, the present invention has been made to solve the above-mentioned problems, and it is an object of the present invention to provide a thread inserting device that is capable of using a cog thread connected to a plurality of needles to thus allow the cog thread to be inserted into a surgical region at different angles and capable of allowing a plurality of cog threads to pullingly anchor to each other to thus improve a lifting effect.

It is another object of the present invention to provide a thread inserting device that is capable of having a guide thread adapted to prevent a thread wound on a needle from being easily unwound from the needle.

The technical problems to be achieved through the present invention are not limited as mentioned above, and other technical problems not mentioned herein will be obviously understood by one of ordinary skill in the art through the following description.

### Technical Solution

To accomplish the above-mentioned objects, according to one aspect of the present invention, there is provided a thread inserting device including a first needle and a second needle each having an opening formed therein extendingly in a longitudinal direction thereof so as to allow a thread to be inserted therethrough and a distal end sharply configured to be inserted into a human body; and a first handle and a second handle coupled to the other end of the first needle and the other end of the second needle, respectively, wherein the thread extends from the distal end of the first needle to the distal end of the second needle through the openings. According to the present invention, desirably, the thread may include a first extension portion locked onto the distal end of the first needle, a fourth extension portion locked onto the distal end of the second needle, a second extension portion extending from the first extension portion and having a plurality of thorns slant toward the fourth extension portion, and a third extension portion extending from the second extension portion and having a plurality of thorns slant toward the second extension portion.

According to the present invention, desirably, the thread may include a fifth extension portion with no thorns formed between the second extension portion and the third extension portion.

According to the present invention, desirably, the thread inserting device may further include lockers adapted to prevent the first extension portion and the fourth extension portion locked onto the distal ends of the first needle and the second needle from escaping from the first needle and the second needle.

According to the present invention, desirably, the thread inserting device may further include a holder adapted to fix the first handle and the second handle thereto to allow the first handle and the second handle to escape therefrom individually.

To accomplish the above-mentioned objects, according to another aspect of the present invention, there is provided a thread inserting device including a needle having an opening formed therein extendingly in a longitudinal direction thereof so as to allow a thread to be inserted therethrough and a distal end sharply configured to be inserted into a human body, a handle coupled to the other end of the needle and communicating with the opening of the needle, the thread having a first extension portion inserted through the opening of the needle and a second extension portion having one end extending from the first extension portion and extendingly wound on the outer peripheral surface of the needle in the longitudinal direction of the needle, and a guide thread having a first guide portion tied to the other end of the second extension portion through a first knot, inserted through the opening on the distal end of the needle, and extending in the longitudinal direction of the needle and a second guide portion extending from the first guide portion, discharged behind the handle, and tied to the outer peripheral surface of the needle through a second knot.

To accomplish the above-mentioned objects, according to still another aspect of the present invention, there is provided a thread inserting device including a needle having an opening formed therein extendingly in a longitudinal direction thereof so as to allow a thread to be inserted therethrough and a distal end sharply configured to be inserted into a human body, a handle coupled to the other end of the needle and communicating with the opening of the needle, a slider fitted onto the needle so as to be movable along the needle, the thread having a first extension portion inserted into the opening of the needle and a second extension portion having one end extending from the first extension portion, extendingly wound on the outer peripheral surface of the needle in the longitudinal direction of the needle, and allowing the other end thereof to be tied to the needle through a third knot, and a guide thread having a first guide portion tied to the outer peripheral surface of the needle through a fourth knot, inserted through the opening of the needle, and extending in the longitudinal direction of the needle and a second guide portion extending from the first guide portion, discharged behind the handle, and tied to the outer peripheral surface of the needle through a second knot.

### Advantageous Effects

According to the present invention, the thread inserting device makes use of two needles to solve the conventional problem wherein surgery is performed using a single needle to cause inserted threads to be overlaid on top of each other.

Further, the thread inserting device according to the present invention is configured to allow the plurality of cog threads to pullingly anchor to each other, thereby improving the lifting effect.

In addition, the thread inserting device according to the present invention is configured to prevent the thread wound on the needle from being unwound from the needle through the guide thread even though the thread inserting device is removed so as to change an insertion position or direction after inserted into the surgical region.

Besides, the thread inserting device according to the present invention is configured to avoid the thread wound on the needle from being unwound from the needle through the guide thread, so that the needle can be inserted repeatedly several times into the surgical region, thereby making it possible to perform drilling surgery for skin peeling.

Additionally, the thread inserting device according to the present invention is configured to have the slider fitted to the needle to allow even the knotted portion of the thread to be easily inserted into the human body.

The effectiveness of the invention is not limited as mentioned above, and it should be understood to those skilled in the art that the effectiveness of the invention may include another effectiveness as not mentioned above from the detailed description of the present invention.

### DESCRIPTION OF DRAWINGS

FIG. 1 is a perspective view showing a thread inserting device according to an embodiment of the present invention.

FIG. 2 is an enlarged view showing a thread of the thread inserting device according to the present invention.

FIG. 3 is an enlarged view showing another example of the thread of the thread inserting device according to the present invention.

FIGS. 4a and 4b are sectional views showing use states of the thread inserting device according to the present invention.

FIG. 5 is a perspective view showing a state where threads anchor to the shape of X under the skin in the thread inserting device according to the present invention.

FIG. 6 is a perspective view showing the thread inserting device according to the present invention, wherein a holder and lockers are further provided.

FIG. 7 is a perspective view showing a thread inserting device according to another embodiment of the present invention.

FIG. 8 is a sectional view showing the thread inserting device according to another embodiment of the present invention.

FIG. 9 is a sectional view showing positions of knots in the thread inserting device according to the present invention.

FIG. 10 is a sectional view showing an auxiliary handle coupled to the thread inserting device according to the present invention.

FIG. 11 is a sectional view showing a thread inserting device according to still another embodiment of the present invention.

FIG. 12 is a sectional view showing a thread locking groove formed in the thread inserting device according to the present invention.

### MODE FOR INVENTION

Hereinafter, a configuration, operations, and operating effects of a thread inserting device for a skin lifting treatment according to the present invention will be disclosed with reference to the attached drawings. In the description, the components shown in the drawings may be not indicated or schematically illustrated for the clarity and convenience of the description, and the sizes of the components may be magnified or reduced. In the description, further, the corresponding parts in the embodiments of the present invention are indicated by corresponding reference numerals and the repeated reference numerals on individual drawings may be not denoted.

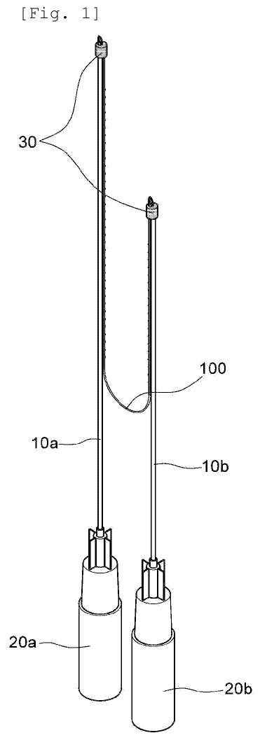

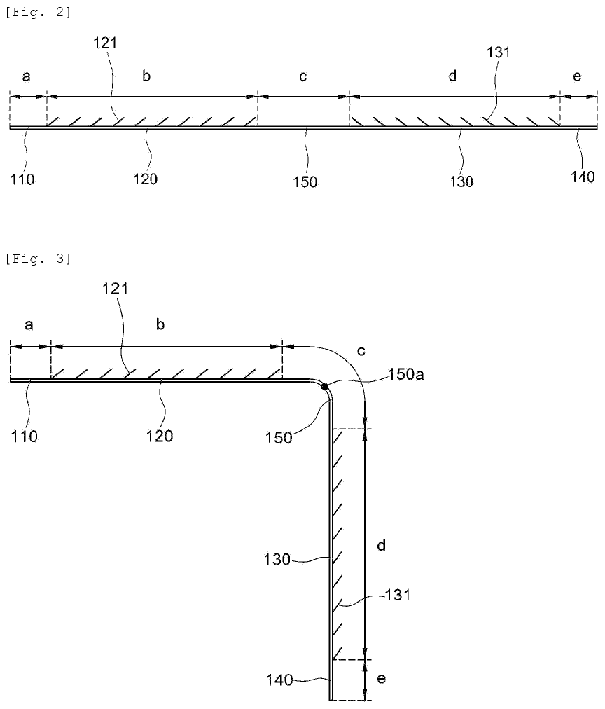

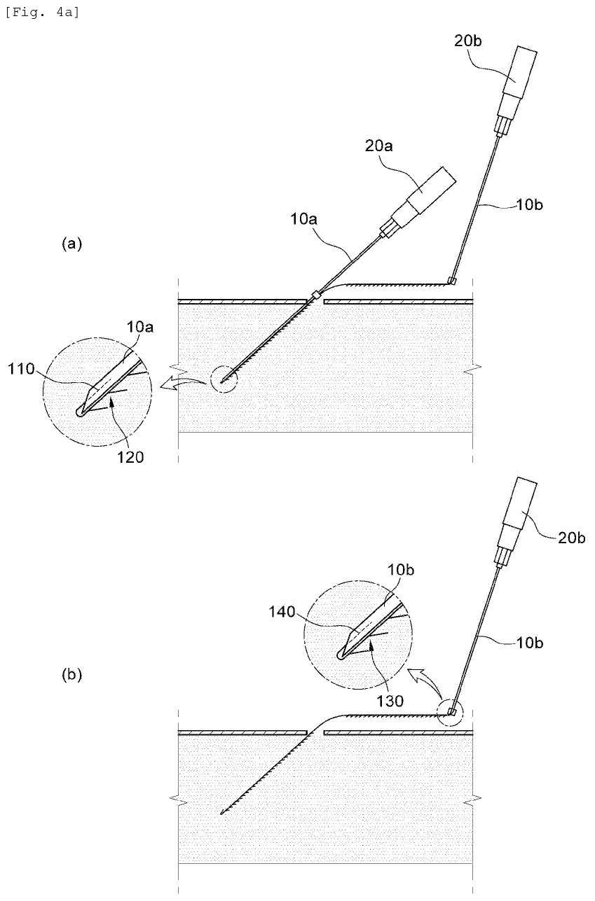

FIG. 1 is a perspective view showing a thread inserting device according to an embodiment of the present invention, and FIGS. 2 and 3 are enlarged views showing a lifting thread of the thread inserting device according to the present invention.

According to an embodiment of the present invention, a thread inserting device includes first and second needles 10a and 10b each having an opening 11 formed therein extendingly in a longitudinal direction thereof so as to allow a thread 100 to be inserted therethrough and a distal end sharply configured to be inserted into a human body; and first and second handles 20a and 20b coupled to the other end of the first and second needles 10a and 10b, respectively, wherein the thread 100 extends from the distal end of the first needle 10a to the distal end of the second needle 10b through the openings 11.

Unlike the conventional surgery in which a single thread inserting needle is used, according to the embodiment of the present invention, a plurality of thread inserting needles are utilized for skin lifting surgery, and a detailed explanation will be given below. First, the first and second needles 10a and 10b have sharp distal ends insertable into the human body and the other end coupled to the first and second handles 20a and 20b, respectively. The first and second needles 10a and 10b have the openings 11 formed therein extendingly in the longitudinal directions thereof to insert the thread 100 therethrough. In this case, the first and second needles 10a and 10b have the same configuration and functions as each other, but may have different lengths or thicknesses from each other, so that their length or thickness may be appropriately varied according to surgery purposes or situations. The first and second handles 20a and 20b are coupled to the other end of the first and second needles 10a and 10b having the sharp distal ends, respectively, and they are held by a surgeon's hands. The first and second handles 20a and 20b are press-fitted to the first and second needles 10a and 10b to help the surgeon perform the skin lifting surgery more accurately.

To connect the two first and second thread needles 10 and 10b to each other, the thread 100 extends from the distal end of the first needle 10a to the distal end of the second needle 10b through the openings 11. In specific, the thread 100 in the conventional practice is configured to allow a portion thereof to be fitted onto the outer periphery of the needle and to allow the other portion thereof to extend along the internal opening 11 of the needle in the longitudinal direction of the needle, thus resulting in inserting the needle under the skin, together with the thread. According to the present invention, however, the thread 100 extends from the distal end of the first needle 10a to the distal end of the second needle 10b through the openings 11b to thus connect the two needles 10a and 10b to each other.

FIG. 6 is a perspective view showing the thread inserting device according to the present invention, wherein a holder and lockers are further provided.

According to desirable characteristics of the present invention, further, the thread inserting device includes a holder 40 adapted to fix the first and second handles 20a and 20b thereto to connect or separate the first needle 10a and the second needle 10b to each other as a single component and from each other individually. The holder 40 serves to connect the first and second handles 20a and 20b coupled to the other end of the first and second needles 10a and 10b to each other and includes a body 41 for supporting the first and second handles 20a and 20b, a housing 43 disposed in the body 41 to surround the first and second handles 20a and 20b, and a cap 45 surroundedly disposed on the outsides of the first and second needles 10a and 10b. Accordingly, the two thread needles can be provided as a single product through the holder 40, and upon surgery, one thread needle is detached from the holder 40 and used. After the use of the first thread needle is finished, the second thread needle is detached from the holder 40 and is used for the surgery.

According to desirable characteristics of the present invention, further, the thread inserting device includes the cap 45 coupled to the first and second handles 20a and 20b to protect the thread 100 that is not inserted into the openings 11 of the first and second needles 10a and 10b and is thus exposed to the outside from the outside. To individually protect the thread 100 not inserted into the openings 11 of the first and second needles 10a and 10b and exposed to the outside, that is, the areas of the thread 100 corresponding to a second extension portion 120, a third extension portion 130, and a fifth extension portion 150, the cap 45 is coupled over the first and second handles 20a and 20b.

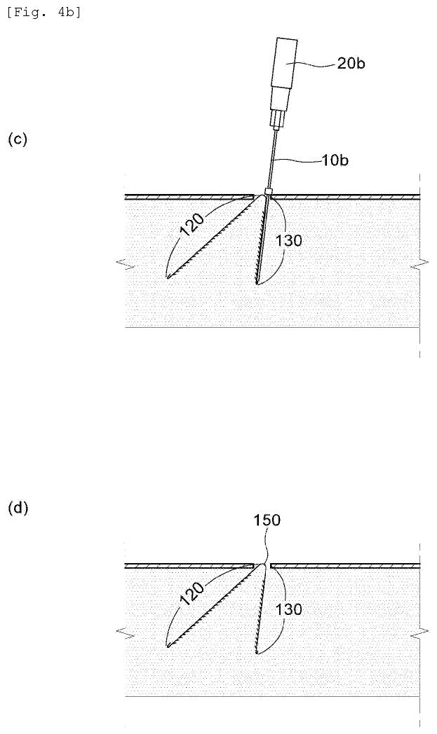

FIGS. 4a and 4b are sectional views showing use states of the thread inserting device according to the present invention.

Under the above-mentioned configuration, now, an explanation of a surgery method will be given. First, the first needle 10a is detached from the holder 40, and the first needle 10a is then inserted into a surgical region, together with the thread 100. Next, if the first handle 20a pulls to remove the first needle 10a, only the first needle 10a is removed in the state where the thread 100 remains in the tissue of skin. In this case, the thread 100 is provided connectedly to the first needle 10a and the second needle 10b, and if the surgery is performed using the first needle 10a, the thread 100 hangs on the distal end of the second needle 10b after escaping from the first needle 10b in the state where half the total length thereof is inserted into the tissue of the skin. Next, the second needle 10b is inserted into the same surgical region in a different direction from the first insertion direction. Accordingly, the remaining area of the thread 100, which is not inserted into the tissue of skin, is inserted under the skin, together with the second needle 10b, and if the second needle 10b is removed, after that, the thread 100 remains in a patient's body. The thread 100 is a cog thread having a plurality of thorns slant in a longitudinal direction thereof. If the thread 100 pulls in the state of being inserted into the tissue of skin, the skin is tightened to reduce wrinkles on the skin.

According to desirable characteristics of the present invention, further, the thread 100 includes a first extension portion 110 locked onto the distal end of the first needle 10a, a fourth extension portion 140 locked onto the distal end of the second needle 10a, the second extension portion 120 extending from the first extension portion 110 and having a plurality of thorns 121 slant toward the fourth extension portion 140, and the third extension portion 130 extending from the second extension portion 120 and having a plurality of thorns 131 slant toward the second extension portion 120.

As shown in FIG. 2, the thread 100 applied to the plurality of needles according to the present invention is divided into five extension portions in a longitudinal direction thereof. First, the first extension portion 110 forms one end of the thread 100 and is locked onto the opening formed on the distal end of the first needle 10a and the fourth extension portion 140 forms the other end of the thread 100 and is locked onto the opening formed on the distal end of the second needle 10a. The first extension portion 110 and the fourth extension portion 140 may have thorns on the surfaces thereof like the cog thread or may have no thorns like a general thread.

Next, the second extension portion 120 extends from the first extension portion 110 and has the plurality of thorns 121 formed slantly thereon toward the fourth extension portion 140. If the thread 100 is inserted into the surgical region, together with the first needle 10a, as shown in FIGS. 4a and 4b, the thorns 121 formed on the second extension portion 120 become slant toward the opposite direction to the insertion direction so that they can be easily inserted into the surgical region. If the first needle 10a is removed, the thread 100 pulls the tissue of skin, while being prevented from easily escaping from the surgical region owing to the thorns 121 of the second extension portion 120, thereby tightening the skin to reduce the wrinkles on the skin. In this case, the length of the second extension portion 120 is desirably equal to the inserted length of the first needle 10a under the skin.

Further, the third extension portion 130 extends from the second extension portion 120 and has the plurality of thorns 131 slant toward the second extension portion 120.

The thorns 131 formed on the third extension portion 130 become slant toward the opposite direction to the direction of the thorns 121 of the second extension portion 120, and because the thread 100 is locked on both of the first needle 10a and the second needle 10b, the third extension portion 130 extending from the fourth extension portion 140 locked onto the distal end of the second needle 10b has the thorns 131 slant toward the second extension portion 120, so that as the second needle 120 is inserted into the same surgical region, the thread 100 can be easily inserted into the surgical region through the thorns 131 slant in the opposite direction to the insertion direction. After that, if the second needle 10b is removed, the thread 100 pulls the tissue of skin, while being prevented from easily escaping from the surgical region owing to the thorns 131 of the third extension portion 130, thereby tightening the skin to reduce the wrinkles on the skin.

In this case, an angle between the first extension portion 110 and the fourth extension portion 140 of the thread 100 inserted into the surgical region is in the range of 0 to 180° according to the insertion directions of the first and second needles 10a and 10b, and like this, if the skin lifting is performed using the inserted thread 100, the lifting result may be efficiently exhibited through the inserted thread in various directions. Besides, the problem occurring in the conventional practice wherein surgery is performed using a single thread inserting needle, that is, the problem in which threads are overlaid on top of each other can be advantageously solved.

According to the present invention, the thread 100 further includes a fifth extension portion 150 with no thorns formed between the second extension portion 120 and the third extension portion 130.

The second extension portion 120 and the third extension portion 130 have the thorns 121 and 131 slant toward different directions from each other, and to distinguish the second extension portion 120 and the third extension portion 130, particularly, to adjust an insertion position of the second needle 10b after the insertion of the first needle 10a, the fifth extension portion 150 is located between the second extension portion 120 and the third extension portion 130. Accordingly, in the case where an insertion length of the thread 100 using the first needle 10a and the second needle 10b is determined in the process of the surgery, the lengths of the second extension portion 120 and the third extension portion 130 corresponding to the determined insertion length of the thread 100 are determined, and next, the fifth extension portion 150 with no thorns is located between the second extension portion 120 and the third extension portion 130. In this case, the fifth extension portion 150 has a bent portion 150a where the thread 100 is bent. Because the insertion length of the thread 100 is not accurately recognized when the surgery is performed using the first needle 10a, disadvantageously, the thread 100 has to be inserted using the second needle 10b under the prediction of the surgeon after the thread 100 has been inserted by a given length. As shown in FIG. 3, the bent portion 150a is formed desirably on the center of the thread 100, more desirably on the center of the fifth extension portion 150, thereby improving the accuracy in the surgery.

According to the present invention, further, the thread inserting device includes lockers 30 adapted to prevent the first extension portion 110 and the fourth extension portion 140 locked onto the distal ends of the first needle 10a and the second needle 10b from escaping from the first needle 10a and the second needle 10b.

To prevent the thread 100 locked onto the distal ends of the first needle 10a and the second needle 10b, particularly the first extension portion 110 and the fourth extension portion 140 locked onto the distal ends of the first needle 10a and the second needle 10b from escaping from the first needle 10a and the second needle 10b, the lockers 30 are additionally provided. The lockers 30 are made of a sponge-like material and fitted to the distal ends of the first needle 10a and the second needle 10b to couple the thread 100 to the first needle 10a and the second needle 10b. Upon the surgery, the lockers 30 are removed so that the first extension portion 110 and the fourth extension portion 140 are inserted under the skin in the state of being locked onto the distal ends of the first needle 10a and the second needle 10b.

According to the present invention, portions of the first extension portion 110 and the fourth extension portion 140 are inserted through the openings 11 of the first needle 10a and the second needle 10b, respectively, the remaining portions of the first extension portion 110 and the fourth extension portion 140 are exposed to the outside from the first needle 10a and the second needle 10b, and the second extension portion 120, the third extension portion 130, and the fifth extension portion 150 are exposed to the outside from the first needle 10a and the second needle 10b.

Only the portions of the first extension portion 110 and the fourth extension portion 140 in length are inserted lockedly through the openings 11 of the first needle 10a and the second needle 10b, and the remaining portions of the first extension portion 110 and the fourth extension portion 140, the second extension portion 120, the third extension portion 130, and the fifth extension portion 150 are exposed to the outside from the first needle 10a and the second needle 10b, so that if the thread 100 is inserted into the surgical region, in this case, a crossing portion of the thread 100 can be minimized.

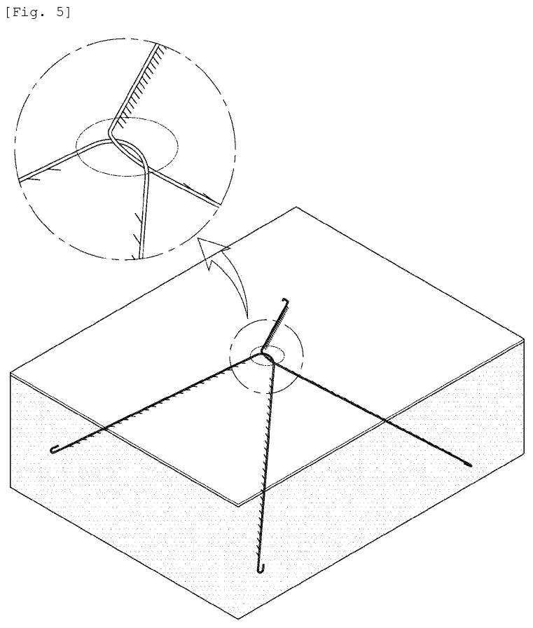

FIG. 5 is a perspective view showing a state where threads anchor to the shape of X under the skin in the thread inserting device according to the present invention.

The inserted thread 100 using the thread inserting device is located with a given angle into the tissue of skin of the surgical region, and in this case, a plurality of threads 100 may be inserted using the plurality of thread inserting needles, thereby improving a lifting effect. For example, one set of the first and second needles 10a and 10b are prepared for the surgery, and accordingly, two sets are prepared to cross the two threads 100. In specific, as shown in FIG. 5, if the two threads 100 are laid on top of each other to the shape of X (that is, to have X-shaped anchoring), the threads 100 with the thorns cross each other and thus anchor to each other to cause strong coupling therebetween, thereby more improving the lifting effect.

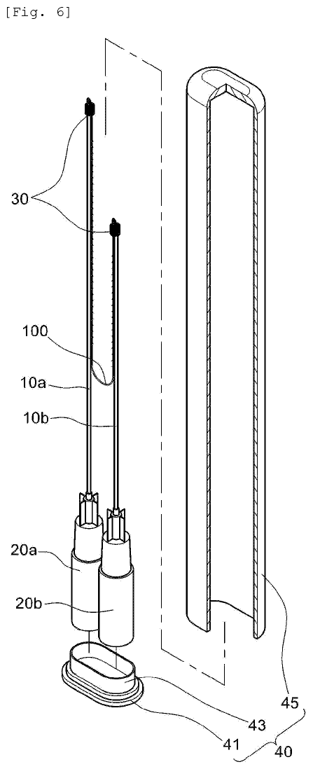

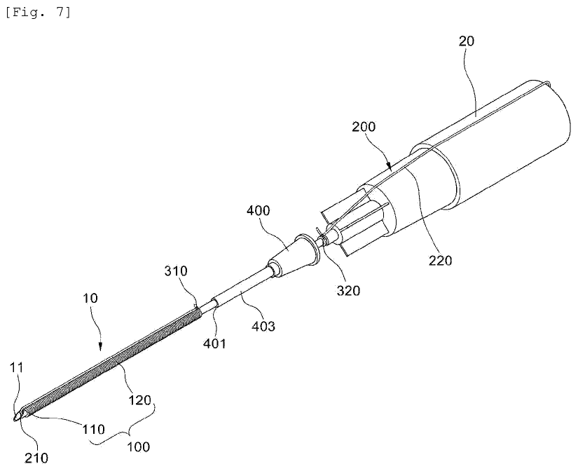

FIG. 7 is a perspective view showing a thread inserting device according to another embodiment of the present invention, and FIG. 8 is a sectional view showing the thread inserting device according to another embodiment of the present invention.

According to another embodiment of the present invention, a thread inserting device includes a needle 10 having an opening 11 formed extendingly therein in a longitudinal direction thereof so as to allow a thread 100 to be inserted therethrough and a distal end sharply configured to be inserted into a human body; a handle 20 coupled to the other end of the needle 10 and communicating with the opening 11 of the needle 10; the thread 100 having a first extension portion 110 inserted into the opening 11 of the needle 10 and a second extension portion 120 having one end extending from the first extension portion 110 and extendingly wound on the outer peripheral surface of the needle 10 in the longitudinal direction of the needle 10; and a guide thread 200 having a first guide portion 210 tied to the other end of the second extension portion 120 through a first knot 310, inserted through the opening 11 on the distal end of the needle 10, and extending in the longitudinal direction of the needle 10 and a second guide portion 220 extending from the first guide portion 210, discharged behind the handle 20, and tied to the outer peripheral surface of the needle 10 through a second knot 320.

In more specific, the needle 10 has the distal end sharply configured to be inserted into the human body and the other end coupled to the handle 20. The needle 10 has the opening 11 formed therein extendingly in the longitudinal direction thereof so as to allow the thread 100 to be inserted therethrough. Further, the handle 20 is coupled to the other end of the needle 10 so as to be held by a surgeon's hand. The handle 20 is press-fitted to the needle to help the surgeon perform skin lifting surgery more accurately. Also, the handle 20 has a through hole formed therein in a longitudinal direction thereof to communicate with the opening 11 of the needle 10. Accordingly, the thread 100 is inserted into the opening 11 from the distal end of the needle 10, passes through the through hole of the handle 20, and extends behind the handle 20.

The thread 100 has the first extension portion 110 inserted into the opening 11 of the needle 10 and the second extension portion 120 having one end extending from the first extension portion 110 and extendingly wound on the outer peripheral surface of the needle 10 in the longitudinal direction of the needle 10.

The first extension portion 110 has a given length so that when the thread 100 is inserted under the skin, the first extension portion 110 does not escape from the opening 11 of the needle 10. The second extension portion 120 extends from the portion of the first extension portion 110 exposed to the outside from the opening 11 and is wound extendingly on the outer peripheral surface of the needle 10 in the longitudinal direction of the needle 10. The second extension portion 120 is extendingly wound on the outer peripheral surface of the needle 10 by a given length, that is, by a desired length to be inserted under the skin, and in this case, the second extension portion 120 may be turned tightly or at given intervals.

In this case, even though the thread 100 does not easily escape from the needle 10 through the first extension portion 110 inserted through the opening 11 of the needle 10 and the second extension portion 120 extendingly wound on the outer peripheral surface of the needle 10, the wound thread 100 may be unwound from the needle 10 if the thread inserting device is removed so as to change an insertion position or direction after inserted into the surgical region. To avoid the problem from occurring, the guide thread 200 is provided to thus prevent the thread 100 from being unwound from the needle 10 and to perform the insertion of the thread 100 after skin peeling for the surgical region has been performed through drilling several times.

The guide thread 200 has the first guide portion 210 tied to the other end of the second extension portion 120 through the first knot 310, inserted into the opening 11 through the distal end of the needle 10, and extending in the longitudinal direction of the needle 10 and the second guide portion 220 extending from the first guide portion 210, discharged behind the handle 20, and tied to the outer peripheral surface of the needle 10 through the second knot 320.

The thread 100 is easily unwound from the needle 10 unless the end of the second extension portion 120 is knotted, and to prevent the thread 100 from being unwound from the needle 10, accordingly, the first guide portion 210 is tied to the second extension portion 120, that is, to the other end of the second extension portion 120 extendingly wound through the first knot 310. After the formation of the first knot 310, the first guide portion 210 extends to the distal end of the needle 10, inserted into the opening 11, and extends toward the handle 20. The second guide portion 220 extending from the first guide portion 210 discharged behind the handle 20 is tied to the outer peripheral surface of the needle 10 through the second knot 320. Through the knotting of the second guide portion 220, the guide thread 200 is fixed without any escape, and even when the surgery is performed using the needle 10, the thread 100 is fixed by means of the guide thread 100, without being unwound from the needle 10. Further, if the thread 100 is inserted into the surgical region after drilling surgery has been performed, the first knot 310 is just untied to remove the needle 10, and accordingly, only the needle 10 is removed in the state where the thread 100 is inserted into the surgical region.

According to the present invention, the thread inserting device includes a slider 400 fitted onto the needle 10 so as to be movable along the needle 10 and having a head 401 disposed on the front end thereof and a shaft 403 extending from the head 401 to constitute a body thereof.

The slider 400 fits onto the needle 10 so as to be movable along the needle 10, and desirably, the slider 400 is located between the second extension portion 120 and the handle 20. Further, the slider 400 slides linearly along the needle 10 forwardly or backwardly. In this case, if the surgery using the needle 10 is performed on the surgical region, a portion of the head 401 of the slider 400 is inserted into the surgical region, together with the needle 10, and accordingly, the slider 400 serves to insert the thread 100 into the human body so that the first knot 310 or a third knot 330 as will be discussed later is not exposed to the outside of the skin. Further, the first knot 310 supportedly comes into contact with the head 401 to thus prevent the second extension portion 120 from being pushed toward the slider 400. The slider 400 has the shaft 403 extending from the head 401 to constitute the body thereof and further may a grasping portion formed on the rear end of the shaft 403 to be easily grasped by the surgeon.

FIG. 9 is a sectional view showing positions of knots in the thread inserting device according to the present invention.

According to desirable characteristics of the present invention, the second knot 320 is tied to one or more positions selected from the position groups consisting of a front end A′ of the head 401, a given portion B of the shaft 403, a given portion C behind the slider 400, a front end C′ of the handle 20, and one end portion D of the needle 10 fixed to the handle 20 after insertedly passing through the handle 20.

The second knot 320 is the knot formed on the outer peripheral surface of the needle 10 and serves to prevent the guide thread 200 from being unwound. In this case, as shown in FIGS. 7 and 8, the second knot 320 may be formed on the front end C′ of the handle 20, but it is not limited thereto. Accordingly, the second knot 320 may be tied to one or more positions selected from the position groups consisting of the front end A′ of the head 401, the given portion B of the shaft 403, the given portion C behind the slider 400, the front end C′ of the handle 20, and one end portion D of the needle 10 fixed to the handle 20 after insertedly passing through the handle 20. In this case, one end portion D of the needle 10 fixed to the handle 20 after insertedly passing through the handle 20 means that the guide thread 200 extendingly passes through the needle 10 and is then fixed to the end of the needle 10 inside the handle 20, and after the guide thread 200 is tied to one end portion D through the second knot 320, accordingly, the needle 10 is fixedly inserted into the handle 20. As the guide thread 200 is fixed through the second knot 320 on various positions, it serves to prevent the thread 100 from being unwound from the needle 10.

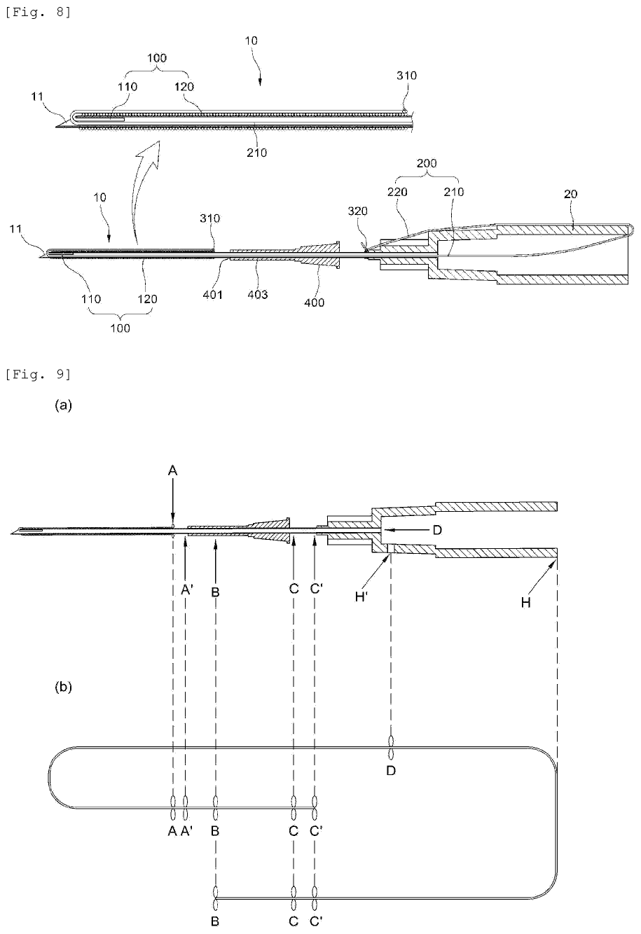

FIG. 10 is a sectional view showing an auxiliary handle coupled to the thread inserting device according to the present invention.

According to desirable characteristics of the present invention, the thread inserting device further includes an auxiliary handle 500 penetratedly coupled to the rear end periphery of the handle 20 to press-fit the second guide portion 220 discharged behind the handle 20 thereto.

The auxiliary handle 500 is press-fitted to the rear end periphery of the handle 20. As mentioned above, the second knot 320 is formed on various positions to fix the second guide portion 220, but through the auxiliary handle 500 just fitted to the rear end periphery of the handle 20 to fix the second guide portion 220, there is no need to form the second knot 320.

FIG. 12 is a sectional view showing a thread locking groove 21 formed in the thread inserting device according to the present invention.

According to desirable characteristics of the present invention, the second guide portion 220 is inserted into an inner portion H′ of a thread locking groove 21 formed on one surface of the handle 20.

The inner portion H′ means that the inner portion of the thread locking groove 21 formed on one surface of the handle 20 and communicating with the through hole of the handle 20 through which the guide thread 200 passes. The thread locking groove 21 serves to allow the second guide portion 220 to be fitted to the handle 20, without formation of a separate knot, so that the second guide portion 220 is grasped by the surgeon's fingers to thus fix the guide thread 200 with ease.

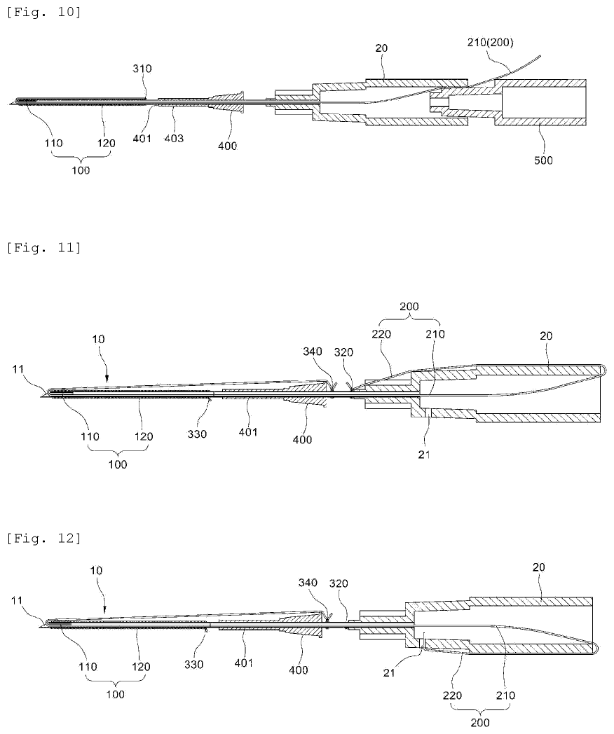

FIG. 11 is a sectional view showing a thread inserting device according to still another embodiment of the present invention.

According to still another embodiment of the present invention, a thread inserting device includes a needle 10 having an opening 11 formed therein extendingly in a longitudinal direction thereof so as to allow a thread 100 to be inserted therethrough and a distal end sharply configured to be inserted into a human body; a handle 20 coupled to the other end of the needle 10 and communicating with the opening 11 of the needle 10; a slider 400 fitted onto the needle 10 so as to be movable along the needle 10; the thread 100 having a first extension portion 110 inserted through the opening 11 of the needle 10 and a second extension portion 120 having one end extending from the first extension portion 110, extendingly wound on the outer peripheral surface of the needle 10 in the longitudinal direction of the needle 10, and allowing the other end thereof to be tied to the needle 10 through a third knot 330; and a guide thread 200 having a first guide portion 210 tied to the outer peripheral surface of the needle 10 through a fourth knot 340, inserted through the opening 11 of the needle 10, and extending in the longitudinal direction of the needle 10 and a second guide portion 220 extending from the first guide portion 210, discharged behind the handle 20, and tied to the outer peripheral surface of the needle 10 through a second knot 320.

According to still another embodiment of the present invention, the thread 100, particularly, the second extension portion 120 of the thread 100 is not tied to the guide thread 200, but it itself forms the third knot 330 on the needle 10 and is thus fixed to the needle 10. Next, the guide thread 200 has the first guide portion 210 tied to the outer peripheral surface of the needle 10 through the fourth knot 340, extending toward the distal end of the needle 10, inserted through the opening 11 of the needle 10, and extending toward the handle 10 and the second guide portion 220 extending from the first guide portion 210, discharged behind the handle 20, and tied to the outer peripheral surface of the needle 10 through the second knot 320.

According to the present invention, desirably, the second knot 320 is tied to one or more positions selected from the position groups consisting of a front end A′ of the head 401, a given portion B of the shaft 403, a given portion C behind the slider 400, a front end C′ of the handle 20, and one end portion D of the needle 10 fixed to the handle 20 after insertedly passing through the handle 20.

According to the present invention, desirably, the fourth knot 340 is tied to one or more positions selected from the position groups consisting of the front end A′ of the head 401, the given portion B of the shaft 403, the given portion C behind the slider 400, and the front end C′ of the handle 20.

As the fourth knot 340 and the second knot 320 are tied on various positions, a mentioned above, the guide thread 200 can be fixed in position. In specific, as the first guide portion 210 forming the fourth knot 340 penetratedly passes through the inside of the needle 10 and extends to thus form the second knot 320, the fourth knot 340 is located at the left side from the second knot 320, that is, on the area close to the opening 11 of the needle 10, thereby preventing the first guide portion 210 and the second guide portion 220 of the guide thread 200 from crossing each other.

Further, each guide portion may be not tied just on a single position, but may be tied on various positions. For example, the first guide portion 210 is tied once on the given portion C behind the slider 400 and tied again on the other end of the second extension portion 120, and the second guide portion 220 is tied once on one end portion D of the needle 10 fixed to the handle 20 after insertedly passing through the handle 20 and tied again on the front end C′ of the handle 20. Through such multiple knots, like this, the fixing effect of the guide thread 200 can be improved.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above teachings. It is therefore intended that the scope of the invention be limited not by this detailed description, but rather by the claims appended hereto. It is therefore evident that the particular embodiments disclosed above may be altered or modified and all such variations are considered within the scope of the disclosed subject matter. Accordingly, the protection sought herein is as set forth in the claims below.

### INDUSTRIAL APPLICABILITY

The present invention is applicable to a skin lifting field through thread insertion.

1. A thread inserting device comprising:
a first needle (10a) and a second needle (10b) each having an opening (11) formed therein extendingly in a longitudinal direction thereof so as to allow a thread (100) to be inserted therethrough and a distal end sharply configured to be inserted into a human body; and
a first handle (20a) and a second handle (20b) coupled to the other end of the first needle (10a) and the other end of the second needle (10b), respectively,
wherein the thread (100) extends from the distal end of the first needle (10a) to the distal end of the second needle (10b) through the openings (11).

2. The thread inserting device according to claim 1, wherein the thread (100) comprises:
a first extension portion (110) locked onto the distal end of the first needle (10a);
a fourth extension portion (140) locked onto the distal end of the second needle (10a);
a second extension portion (120) extending from the first extension portion (110) and having a plurality of thorns (121) slant toward the fourth extension portion (140); and
a third extension portion (130) extending from the second extension portion (120) and having a plurality of thorns (131) slant toward the second extension portion (120).

3. The thread inserting device according to claim 2, wherein the thread (100) comprises a fifth extension portion (150) with no thorns formed between the second extension portion (120) and the third extension portion (130).

4. The thread inserting device according to claim 2, further comprising lockers (30) adapted to prevent the first extension portion (110) and the fourth extension portion (140) locked onto the distal ends of the first needle (10a) and the second needle (10b) from escaping from the first needle (10a) and the second needle (10b).

5. The thread inserting device according to claim 2, wherein portions of the first extension portion (110) and the fourth extension portion (140) are inserted through the openings (11) of the first needle (10a) and the second needle (10b), respectively, the remaining portions of the first extension portion (110) and the fourth extension portion (140) are exposed to the outside from the first needle (10a) and the second needle (10b), and the second extension portion (120), the third extension portion (130), and the fifth extension portion (150) are exposed to the outside from the first needle (10a) and the second needle (10b).

6. The thread inserting device according to claim 1, further comprising a holder (40) adapted to fix the first handle (20a) and the second handle (20b) thereto to allow the first handle (20a) and the second handle (20b) to escape therefrom individually.

7. The thread inserting device according to claim 1 or 6, further comprising a cap (45) coupled to the first handle (20a) and the second handle (20b) to protect the thread (100) that is exposed to the outside, while being not inserted through the openings (11) of the first and second needles (10a) and (10b), from the outside.

8. A thread inserting device comprising:
a needle (10) having an opening (11) formed therein extendingly in a longitudinal direction thereof so as to allow a thread (100) to be inserted therethrough and a distal end sharply configured to be inserted into a human body;
a handle (20) coupled to the other end of the needle (10) and communicating with the opening (11) of the needle (10);
the thread (100) having a first extension portion (110) inserted through the opening (11) of the needle (10) and a second extension portion (120) having one end extending from the first extension portion (110) and extendingly wound on the outer peripheral surface of the needle (10) along the longitudinal direction of the needle (10); and
a guide thread (200) having a first guide portion (210) tied to the other end of the second extension portion (120) through a first knot (310), inserted through the opening (11) on the distal end of the needle (10), and extending in the longitudinal direction of the needle (10) and a second guide portion (220) extending from the first guide portion (210), discharged behind the handle (20), and tied to the outer peripheral surface of the needle (10) through a second knot (320).

9. The thread inserting device according to claim 8, further comprising a slider (400) fitted onto the needle (10) so as to be movable along the needle (10).

10. The thread inserting device according to claim 9, wherein the slider (400) comprises a head (401) disposed on the front end thereof and a shaft (403) extending from the head (401) to constitute a body thereof.

11. The thread inserting device according to claim 10, wherein the second knot (320) is tied to one or more positions selected from the position groups consisting of a front end (A′) of the head (401), a given portion (B) of the shaft (403), a given portion (C) behind the slider (400), a front end (C′) of the handle (20), and one end portion (D) of the needle (10) fixed to the handle (20) after insertedly passing through the handle (20).

12. The thread inserting device according to claim 8, further comprising an auxiliary handle (500) penetratedly coupled to the rear end periphery of the handle (20) to press-fit the second guide portion (220) discharged behind the handle (20) thereto.

13. The thread inserting device according to claim 8 or 12, wherein the second guide portion (220) is inserted into an inner portion (H′) of a thread locking groove (21) formed on one surface of the handle (20).

14. A thread inserting device comprising:
a needle (10) having an opening (11) formed therein extendingly in a longitudinal direction thereof so as to allow a thread (100) to be inserted therethrough and a distal end sharply configured to be inserted into a human body;
a handle (20) coupled to the other end of the needle (10) and communicating with the opening (11) of the needle (10);
the thread (100) having a first extension portion (110) inserted through the opening (11) of the needle (10) and a second extension portion (120) having one end extending from the first extension portion (110), extendingly wound on the outer peripheral surface of the needle (10) along the longitudinal direction of the needle (10), and allowing the other end thereof to be tied to the needle (10) through a third knot (330); and
a guide thread (200) having a first guide portion (210) tied to the outer peripheral surface of the needle (10) through a fourth knot (340), inserted through the opening (11) of the needle (10), and extending in the longitudinal direction of the needle (10) and a second guide portion (220) extending from the first guide portion (210), discharged behind the handle (20), and tied to the outer peripheral surface of the needle (10) through a second knot (320).

15. The thread inserting device according to claim 14, further comprising a slider (400) fitted onto the needle (10) so as to be movable along the needle (10).

16. The thread inserting device according to claim 15, wherein the slider (400) comprises a head (401) disposed on the front end thereof and a shaft (403) extending from the head (401) to constitute a body thereof.

17. The thread inserting device according to claim 16, wherein the second knot (320) is tied to one or more positions selected from the position groups consisting of a front end (A′) of the head (401), a given portion (B) of the shaft (403), a given portion (C) behind the slider (400), a front end (C′) of the handle (20), and one end portion (D) of the needle (10) fixed to the handle (20) after insertedly passing through the handle (20).

18. The thread inserting device according to claim 16, wherein the fourth knot 340 is tied to one or more positions selected from the position groups consisting of the front end (A′) of the head (401), the given portion (B) of the shaft (403), the given portion (C) behind the slider (400), and the front end (C′) of the handle (20).

19. The thread inserting device according to claim 14, wherein the second guide portion (220) is inserted into an inner portion (H′) of a thread locking groove (21) formed on one surface of the handle (20).
